(19) United States
(12) Patent Application Publication
(10) Pub. No.: US 2023/0114233 A1
(43) Pub. Date: Apr. 13, 2023

(54) SYSTEM FOR DETECTING AND QUANTIFYING A PLURALITY OF MOLECULES IN A PLURALITY OF BIOLOGICAL SAMPLES

(71) Applicant: ALGORITHMIC BIOLOGICS PRIVATE LIMITED, Bangalore (IN)

(72) Inventor: Manoj Gopalkrishnan, Mumbai (IN)

(21) Appl. No.: 17/956,870

(22) Filed: Sep. 30, 2022

(30) Foreign Application Priority Data
Sep. 30, 2021 (IN) 202141044465

(51) Int. Cl. G16B 40/20; G06N 7/00

(52) U.S. Cl. CPC G16B 40/20; G06N 7/005

(57) ABSTRACT

There is provided a system for detecting and quantifying a plurality of molecules in a plurality of biological samples based on a noisy output data from an assay on each pool. The system (i) generates a sensing matrix with a plurality of rows (m) and a plurality of columns (n) based on at least one input, (ii) obtains a noisy output data after completing the assay in each pool (iii) generates a probabilistic graphical model based on a non-linear equation, and (iv) detects and quantifies the molecules in the plurality of biological samples by providing the noisy output data from each pool to the probabilistic graphical model and identifying and quantifying the presence of the molecules in the plurality of biological samples by executing exact or approximate Bayesian inference for the probabilistic graphical model along with the noisy output data.

### BACKGROUND

### CROSS-REFERENCE TO PRIOR FILED PATENT APPLICATIONS

This application claims priority from the Indian provisional application no. 202141044465 filed on Sep. 30, 2021, which is herein incorporated by reference.

### TECHNICAL FIELD

Embodiments herein generally relate to a technique for solving inverse problems, and provide a system and method for solving nonlinear inverse problems using a probabilistic graphical model. More particularly, the embodiments herein provide a system and method for detecting and quantifying a plurality of molecules in a plurality of biological samples based on a noisy output data from an assay on each pool. Further, the system and method detect a condition of interest based on the detected and quantified molecules in the plurality of biological samples.

### DESCRIPTION OF THE RELATED ART

Public health screenings are usually cost-sensitive. Most individuals are likely to be negative for condition of interest. They are not being done or not being done in a comprehensive manner in many countries because of the cost factor.

Inverse problems have applications in many branches of science and engineering such as medical diagnostics, agriculture, robotics, optics, geophysics, imaging, acoustics, and civil and mechanical engineering. In a case of “forward problems”, an output (effect or response) is estimated from an input (cause). In contrast, the “inverse problems” require estimating the cause or input parameters from the effect or response (output). The inverse problems are usually classified into two categories that includes linear inverse problems and nonlinear inverse problems.

The linear inverse problems are usually formulated as y = Ax be a column vector obtained by multiplying a matrix A with the vector x. Let x = (x₁, x₂...xₙ)^{T} be a column vector (e.g., of nonnegative real numbers) representing some signal. For example, as i varies from 1 to n, xᵢ might represent a number of copies of some molecular analyte present in an i^{th} sample. Let m « n be another positive integer. A sensing matrix or a pooling matrix A is a matrix of m rows and n columns with all entries as nonnegative real numbers.

Let y′ be a noisy measurement of y. For example, for samples, rows of A describe how to combine the samples into pools. A number yⱼ represents the number of copies of the molecular analyte in the j^{th} pool. A number yⱼ’ represents a measurement of yⱼ, for example by using some molecular diagnostic assay like the quantitative PCR test.

The linear inverse problem is a problem of estimating x from A and y′. When m < n, it admits infinitely many solutions and needs assumptions either about regularity of solution or about prior information to effectively identify a unique solution. One common regularity assumption is sparsity which means that the vector x has very few nonzero entries. Algorithms developed for this setting are known as compressed sensing in signal processing literature, and as sparse regression in statistics literature. The linear inverse problems are well-studied, and very successfully solved.

However, standard algorithms for the linear inverse problems have to be implemented on a computer. Thus, real numbers are represented as floating point numbers in the computer. When a range of nonzero values that one may encounter spans multiple orders of magnitude, then representing this problem on the computer by floating point numbers can lead to numerical inaccuracies. An example case is that measuring numbers of molecules in an assay like quantitative polymerase chain reaction (qPCR). A range of these numbers may vary from one molecule to a trillion molecules.

Further, many real-world inverse problems are nonlinear which have not been fully explored, unlike the linear inverse problems, due to the complexity of the problem. The nonlinear inverse problems are of the type where v = f(u). Given a noisy measurement v′ of v and a function f, one wishes to recover a vector u. The nonlinear inverse problems have been thought of as hopeless. Essentially the only successes in this field have to do with inverse scattering problems.

Therefore, there is a need to address the aforementioned technical drawbacks in existing technologies in solving inverse problems.

### SUMMARY

In view of foregoing embodiments herein provide a system and method for detecting and quantifying a plurality of molecules in a plurality of biological samples based on a noisy output data from an assay on each pool.

In a first aspect, a system for detecting and quantifying a plurality of molecules in a plurality of biological samples based on a noisy output data from an assay on each pool, is provided. The system includes a memory that stores a set of instructions, and a processor that is configured to execute the set of instructions for (i) generating, using a sample decoding device, a sensing matrix with a plurality of rows (m) and a plurality of columns (n) based on at least one input from a user, the plurality of biological samples are combined or grouped based on the sensing matrix to generate a plurality of pools, (ii) obtaining, from a testing machine, a noisy output data after completing the assay in each pool, the noisy output data is an output data with noise from each pool, (iii) generating, using the sample decoding device, a probabilistic graphical model based on a non-linear equation for detecting and quantifying the plurality of molecules in the plurality of biological samples, the non-linear equation is generated based on a plurality of variables that comprise the generated sensing matrix, a plurality of output data of the plurality of pools, and a quantitative measure of each molecule, and (iv) detecting and quantifying, using the sample decoding device, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data. The plurality of variables are converted as conditionals statements in the probabilistic graphical model.

In some embodiments, the processor is configured to detect a condition of interest based on the detected and quantified molecules in the plurality of biological samples. The condition of interest includes at least one of an infectious disease, cancer, a genetic disease, an inflammation condition, a metabolic syndrome, cardiac disease, or diabetes.

In some embodiments, the testing machine is a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy.

In some embodiments, the nonlinear equation comprises v = f(A g(u)), wherein the,
(a) A is the sensing matrix with the plurality of rows (m) and the plurality of columns (n);
(b) u is a column vector of dimension n, wherein the n indicates a number of the plurality of biological samples to be tested, wherein detection of the column vector (u) enables to detect the presence or absence of the plurality of molecules in the plurality of biological samples and quantify the plurality of molecules if the molecules are present in the plurality of biological samples;
(c) v is a vector of dimension m, wherein v is considered as the output data from each pool and v′ is considered as the noisy output data of the output data from each pool;
(d) g is a nonlinear vector-valued function of n variables; and
(e) f is a nonlinear vector-valued function of m variables.

In some embodiments, executing the exact or approximate Bayesian inference includes systemically specifying prior and regulatory conditions for the probabilistic graphical model.

In some embodiments, the processor is configured to (i) convert a noisy linear inverse problem into a noisy nonlinear inverse problem when there are multiples orders of nonzero entries in the sensing matrix, and (ii) construct the nonlinear equation v = log(A e^{u}) by considering f and g as log and exp functions instead of considering f and g as identity functions, wherein the nonzero entries indicate that each sample in the plurality of columns (n) of the sensing matrix (A) represents at least one signal.

In some embodiments, the processor is configured to perform n (n=1,2,3,....) iterations to create the sensing matrix for obtaining compression in pooling by (i) creating a first sensing matrix based on a size of the assay, (ii) subsequently creating a second sensing matrix based on the first sensing matrix, and (iii) thereafter creating a n^{th} sensing matrix based on the second sensing matrix or a previous sensing matrix, wherein a size of the second sensing matrix or a number of pools of the second sensing matrix is smaller than a number of pools of the first sensing matrix.

In some embodiments, (i) the plurality of rows (m) indicate the plurality of pools to be created for testing of the plurality of biological samples; and (ii) the plurality of columns (n) indicate the plurality of biological samples to be tested.

In some embodiments, the at least input includes at least one of a name of the assay, and a size of the assay, and a number of biological samples estimated as positive out of the total number of biological samples. The size of the assay indicates a total number of biological samples to be tested.

In another aspect, a processor implemented method for detecting and quantifying a plurality of molecules in a plurality of biological samples based on a noisy output data from an assay on each pool, is provided. The method includes (i) generating, using a sample decoding device, a sensing matrix with a plurality of rows (m) and a plurality of columns (n) based on at least one input from a user, the plurality of biological samples are combined or grouped based on the sensing matrix to generate a plurality of pools, (ii) obtaining, from a testing machine, a noisy output data after completing the assay in each pool, the noisy output data is an output data with noise from each pool, (iii) generating, using the sample decoding device, a probabilistic graphical model based on a non-linear equation for detecting and quantifying the plurality of molecules in the plurality of biological samples, the non-linear equation is generated based on a plurality of variables that comprise the generated sensing matrix, a plurality of output data of the plurality of pools, and a quantitative measure of each molecule, and (iv) detecting and quantifying, using the sample decoding device, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data. The plurality of variables are converted as conditionals statements in the probabilistic graphical model.

In some embodiments, the method further includes detecting a condition of interest based on the detected and quantified molecules in the plurality of biological samples. The condition of interest includes at least one of an infectious disease, cancer, a genetic disease, an inflammation condition, a metabolic syndrome, cardiac disease, or diabetes.

In some embodiments, the testing machine is a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy.

In some embodiments, the nonlinear equation comprises v = f(A g(u)), wherein the,
(a) A is the sensing matrix with the plurality of rows (m) and the plurality of columns (n);
(b) u is a column vector of dimension n, wherein the n indicates a number of the plurality of biological samples to be tested, wherein detection of the column vector (u) enables to detect the presence or absence of the plurality of molecules in the plurality of biological samples and quantify the plurality of molecules if the molecules are present in the plurality of biological samples;
(c) v is a vector of dimension m, wherein v is considered as the output data from each pool and v′ is considered as the noisy output data of the output data from each pool;
(d) g is a nonlinear vector-valued function of n variables; and
(e) f is a nonlinear vector-valued function of m variables.

In some embodiments, executing the exact or approximate Bayesian inference includes systemically specifying prior and regulatory conditions for the probabilistic graphical model.

In some embodiments, the method further includes (i) convert a noisy linear inverse problem into a noisy nonlinear inverse problem when there are multiples orders of nonzero entries in the sensing matrix, and (ii) construct the nonlinear equation v = log(A e^{u}) by considering f and g as log and exp functions instead of considering f and g as identity functions, the nonzero entries indicate that each sample in the plurality of columns (n) of the sensing matrix (A) represents at least one signal.

In some embodiments, the method performs n (n=1,2,3,....) iterations to create the sensing matrix for obtaining compression in pooling by (i) creating a first sensing matrix based on a size of the assay, (ii) subsequently creating a second sensing matrix based on the first sensing matrix, and (iii) thereafter creating a n^{th} sensing matrix based on the second sensing matrix or a previous sensing matrix, wherein a size of the second sensing matrix or a number of pools of the second sensing matrix is smaller than a number of pools of the first sensing matrix.

In some embodiments, (i) the plurality of rows (m) indicate the plurality of pools to be created for testing of the plurality of biological samples; and (ii) the plurality of columns (n) indicate the plurality of biological samples to be tested.

In some embodiments, the at least input includes at least one of a name of the assay, and a size of the assay, and a number of biological samples estimated as positive out of the total number of biological samples. The size of the assay indicates a total number of biological samples to be tested.

In another aspect, one or more non-transitory computer-readable storage mediums storing the one or more sequences of instructions, which when executed by the one or more processors, causes to perform a method of detecting and quantifying a plurality of molecules in a plurality of biological samples based on a noisy output data from an assay on each pool, are provided.

The embodiments herein are advantageous in that the system and method provide a technically significant approach that accurately detect and quantify, in less time, the presence or absence of the plurality of molecules in the plurality of biological samples from a single-round combinatorial pooling for the assay.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed descriptions with reference to the drawings, in which:

FIG. 1 is a block diagram that illustrates a system for detecting and quantifying a plurality of molecules in a plurality of biological samples according to some embodiments herein;

FIG. 2 is an exemplary block diagram that illustrates a use of the system of FIG. 1 for detecting or retrieving test results of one or more biological samples from a polymerase chain reaction (PCR) according to some embodiments herein;

FIG. 3 is an exemplary block diagram that illustrates a use of the system of FIG. 1 for detecting or retrieving test results of one or more biological samples, where a pooling matrix created from one or more iterations of pooling according to some embodiments herein;

FIG. 4 illustrates a method for detecting and quantifying a plurality of molecules in a plurality of biological samples based on a noisy output data from an assay on each pool according to some embodiments herein;

FIG. 5A is a table of experimental results that illustrates an accuracy of the system of FIG. 1 in detecting and quantifying the plurality of molecules in the plurality of biological samples according to some embodiments herein;

FIG. 5B is a table of experimental results that illustrates a computational efficiency of the system of FIG. 1 in detecting and quantifying the plurality of molecules in the plurality of biological samples according to some embodiments herein;

FIG. 5C is an exemplary graphical representation that illustrates sensitivity of the system of FIG. 1 in detecting and quantifying the plurality of molecules in the plurality of biological samples in comparison with existing linear solver or compressed sensing solver according to some embodiments herein;

FIG. 5D is an exemplary graphical representation that illustrates specificity of the system of FIG. 1 in detecting and quantifying the plurality of molecules in the plurality of biological samples in comparison with existing linear solver or compressed sensing solver according to some embodiments herein;

FIG. 6 is an exemplary 24*64 sensing matrix that is generated using the system of FIG. 1 according to some embodiments herein; and

FIG. 7 is a schematic diagram of computer architecture of a computing device or a molecular computer, in accordance with the embodiments herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

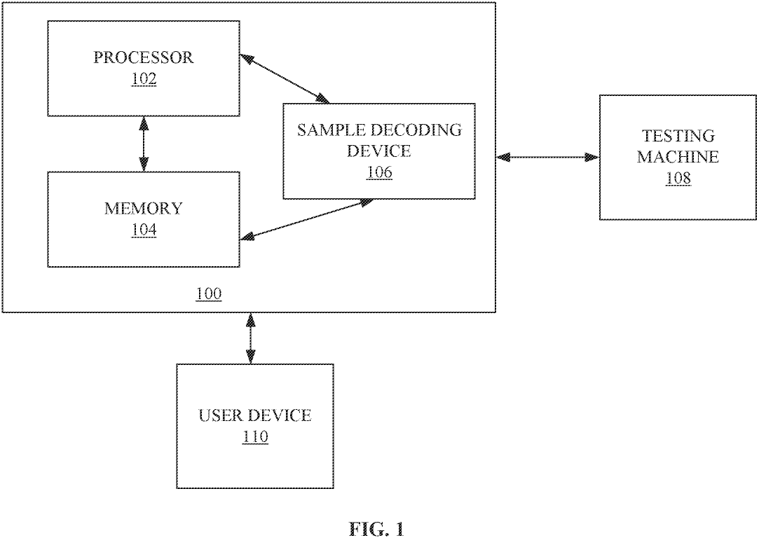

As mentioned, there remains a need for a technique to solve nonlinear inverse problems. The embodiments herein achieve this by providing a system and method for solving nonlinear inverse problems using a probabilistic graphical model. Referring now to the drawings and more particularly to FIGS. 1 through 7, where similar reference characters denote corresponding features consistently throughout the figures, there are shown preferred embodiments.

FIG. 1 is a block diagram that illustrates a system for detecting and quantifying a plurality of molecules in a plurality of biological samples according to some embodiments herein. The system 100 includes a processor 102 and a memory 104 having stored thereon computer-executable instructions that are executable by the processor 102 and that cause the system 100 to (i) generating, using a sample decoding device 106, a sensing matrix with a plurality of rows (m) and a plurality of columns (n) based on at least one input from a user, the plurality of biological samples are combined or grouped based on the sensing matrix to prepare a plurality of pools, (ii) measure, using a testing machine 108, a noisy output data after completing the assay in each pool, (iii) create a nonlinear equation that is defined by

$$v = f ( A g ( u ) )$$

where (a) A is the sensing matrix of m rows and n columns; (b) u is a column vector of dimension n, the n indicates a number of the plurality of biological samples to be tested and detection of the column vector (u) enables to detect the presence or absence of the molecules in the plurality of biological samples and quantify the molecules if the molecules are present in the plurality of biological samples; (c) v is a vector of dimension m, v is considered as the output data from each pool and v′ is considered as the noisy output data from each pool; (d) g is a nonlinear vector-valued function of n variables; and (e) f is a nonlinear vector-valued function of m variables, (ii) generate, using a sample decoding device 106, a probabilistic graphical model based on the nonlinear equation, and (iii) detect and quantify, using a sample decoding device 106, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data. The noisy output data is an output data with noise from each pool. In one example, the noisy output data is Ct values from amplification curves for each pool. Ct values derived from a PCR machine. The assay is an investigative procedure in laboratory medicine, mining, pharmacology, environmental biology and molecular biology for qualitatively assessing or quantitatively measuring the presence, amount, or functional activity of a target entity.

The non-linear equation is generated based on a plurality of variables that comprise the generated sensing matrix, a plurality of output data of the plurality of pools, and a quantitative measure of each molecule. The plurality of variables are converted as conditionals statements in the probabilistic graphical model. The conditional statements enable to make a decision on detection and quantification of molecules based on the inferences executed.

In some embodiments, the probabilistic graphical model is generated, using a probabilistic programming language such as Stan, by (i) writing the nonlinear equation, (ii) the plurality of variables are converted as conditioning statements in Stan, (iii) automatically generating the underlying probabilistic graphical model, using probabilistic programming language interpreter/ compiler from the code specification. The observed values for the conditioned variables are fed at a time of exact or approximate Bayesian inference such as Markov Chain Monte Carlo inference algorithms.

The nonlinear functions (f, g) may be at least one of, but not limited to, (log, exp), (softmax, identity), (RELU, identity), or (tanh, identity) applied to each component of the argument vector.

The probabilistic graphical model allows specification of prior information and regularity conditions in a systematic way to solve the nonlinear equation. For example, one regularity condition is sparsity. Another regularity condition is when most entries have a numerical value below a threshold, and very few entries have a numerical value much above the threshold. This kind of regularity is seen, for example, in mass spectrometry data measuring metabolite levels in blood samples. The few samples that have high numerical value can correspond to an abnormally high value of a metabolite, indicating a disease state. In this way, the probabilistic graphical model allows modeling and exploitation of other kinds of regularity condition than just sparsity.

In some embodiments, the system 100 enables to solve linear inverse problems when f and g are identity functions.

The class of nonlinear inverse problems described above may also be interpreted as a single layer in a neural network where a firing pattern of the n input nodes i is identified from a firing pattern of the output nodes. Such layers may be composed, so that a sequence of such relations includes:

$${\text{v}}_{1} = \text{f} \left({\text{A}}_{01} \text{g} \left(\text{u}\right)\right)$$

$${\text{v}}_{2} = \text{f} \left({\text{A}}_{12} \text{g} \left({\text{v}}_{1}\right)\right)$$

$${\text{v}}_{3} = \text{f} \left({\text{A}}_{23} \text{g} \left({\text{v}}_{2}\right)\right)$$

$${\text{v}}_{\text{d}} = \text{f} \left(\text{Ad-1,d g} \left({\text{v}}_{\text{d-1}}\right)\right)$$

The system 100 estimates the column vector (u) given all matrices A_{1-1,1} for layers 1 = 1 to d, the nonlinear functions (f, g) and a noisy measurement v_{dʹ} of v_{d} by running suitable Markov Chain Monte Carlo inference algorithms for the probabilistic graphical model.

In some embodiments, the sample decoding device 106 generates the sensing matrix based on the at least one input that includes a name of the assay, and a size of the assay, wherein the size of the assay indicates a total number of biological samples to be tested and a number of biological samples estimated as positive out of the total number of biological samples. The at least one input may be given via a user device 110 by the user.

In some embodiments, the testing machine is a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy.

The system 100 runs exact or approximate Bayesian inference, using at least one technique that includes Markov chain Monte Carlo (MCMC), variational inference, message passing, or exact inference.

In some embodiments, the biological samples may be, but not limited to, a blood sample, a urine sample, a saliva sample, a swab sample, any biofluid or bodily fluid, any tissue sample, a tooth sample, a sweat sample, a nail sample, a skin sample, a hair sample, or a fecal sample. The molecules may include, but not limited to, infectious agents or microbial analytes or disease-causing agents or pathogens, contamination agents, blood analytes, chemical species or chemical substances, proteins, nucleic acids, alleles, marker regions and any biomolecules. The infectious agents may include, but not limited to, virus, bacteria, fungi, protozoa and helminth. The chemical species may include, but not limited to, sodium (Na), potassium (K), urea, glucose, and creatinine. The chemical species or chemical substance is a substance that is composed of chemically identical molecular entities. The proteins are biomolecules comprised of amino acid residues joined together by peptide bonds. The protein may include, but is not limited to, antibodies, enzymes, hormones, transport proteins, and storage proteins. The nucleic acids include deoxyribonucleic acid (DNA), ribonucleic acid (RNA), or peptide nucleic acid (PNA). Biomolecules are any molecules that are produced by cells and living organisms. The number of tests may be a number of multiplexed tests.

The system 100 may be at least one of a cloud computing device (may be a part of a public cloud or a private cloud), a server, or a computing device. The server may be at least one of a standalone server, a server on a cloud, or the like. The computing device may be, but is not limited to, a personal computer, a notebook, a tablet, desktop computer, a laptop, a handheld device, a mobile device, or the like. Also, the system 100 may be at least one of, a microcontroller, a processor, a System on Chip (SoC), an integrated chip (IC), a microprocessor based programmable consumer electronic device, and so on. The system 100 may be connected with user devices using a communication network. Examples of the communication network may be, but are not limited to, Internet, a wired network, a wireless network (a Wi-Fi network, a cellular network, a Wi-Fi Hotspot, Bluetooth, or Zigbee) and the like).

The system 100 is further configured to detect a condition of interest based on the detected and quantified molecules in the plurality of biological samples, wherein the condition of interest comprises at least one of an infectious disease, cancer, a genetic disease, an inflammation condition, a metabolic syndrome, cardiac disease, or diabetes.

The system 100 may be used to solve nonlinear inverse problems in medical diagnostics assay, agriculture, robotics, optics, geophysics, imaging, acoustics, and civil and mechanical engineering.

In one exemplary embodiment, the system 100 is used for recovering individual sample results from single-round combinatorial pooling for quantitative polymerase chain reaction (qPCR). Consider an example scenario, where biological samples that have been tested may be numbered as 1,2,3........n and indexed by ‘i’, and the pools or tests created for the biological samples may be numbered as 1,2,3........n and indexed by ‘j’. In such scenario, the inverse problem is a noisy linear inverse problem.

In existing approaches, a compressed sensing method is used to solve the noisy linear inverse problem by constructing a noisy linear equation by considering converted quantitative measure of viral load or microbial load of each of the pools (that are positive) and a pooling matrix created for the testing of the biological samples. However, when each component of the vector or more components of the vector include nonzero values, the existing approaches may lead to inaccuracies in the test results.

Hence, the system 100 (i) converts the noisy linear inverse problem into a noisy nonlinear inverse problem by choosing f and g to be log and exp instead of identity functions, where log(x) is understood as (log(x₁), log(x₂), ..., log(xₙ)), defining u_i := log x_i that yields y = Ae^{u}, then taking log on both sides, and defining v = log(y) that yields the nonlinear inverse problem

$$\text{v = log} \left({\text{A e}}^{\text{u}}\right)$$

(ii) solves the nonlinear inverse problem by specifying a regularity condition on u after receiving a noisy measurement v′ of v, a matrix A, and functions f and g, to determine status or results of each biological samples that have been used for testing. If the regularity condition was sparsity on x, this may be modelled as a Laplace prior on each component of u centered at a sufficiently large negative value, and with a carefully tuned variance. The results of the biological sample may indicate whether viruses or microbes are present in the biological sample or not and a viral load or a microbial load of the biological samples, if the viruses or microbes are present in the biological samples. Thus, the biological samples may be tested in a single round of testing without a need for a second confirmatory round.

In another exemplary embodiment, the system 100 is used for public health PCR-based and Nucleic Acid Testing-based screening for (i) identifying infectious diseases such as Covid19 or Tuberculosis or Ebola or HIV etc., (ii) detecting oncomarkers such as Human Pappiloma Virus or cell-free DNA/ circulating tumor DNA for early cancer detection, (iii) detecting markers indicating inflammation, metabolic syndrome, cardiac disease, diabetes, etc.

In another exemplary embodiment, the system 100 is used for blood transfusion safety testing which is done to ensure that a blood transfusion recipient does not inadvertently receive blood containing HIV or Hepatitis or similar dangerous pathogens. While the Nucleic Acid Tests (NAT) are the gold standard, due to cost reasons in low median income countries the less accurate ELISA and Immunoassay tests are used. This leads to a public health crisis especially among populations at high risk due to constant transfusions, e.g., Thalassemic children. The system 100 allows making NAT testing more affordable, thus unlocking wider deployment of this test, and safer blood transfusion for all.

Further, public health next generation sequencing-based screening can reveal which individuals are at greater risk of various conditions like cardiac disease, neurological disorders, etc., and allow for actionable information that can enhance lifespan as well as wellness. The cost of such screening programs can be dramatically reduced by using the present disclosure, allowing for adoption of such public health screening in more countries across the world.

Similarly, public health mass spectrometry-based screening can reveal newborns at risk of mortality and morbidity due to inborn errors of metabolism. The present disclosure makes this screening affordable, and hence capable of comprehensive deployment in many countries.

Further, the present disclosure leads to following practical applications in agriculture becoming more affordable such as (i) Screening plants for pathogens: E.g., Orange trees can have a bacterium called orange canker. Identifying the infected trees very early is key to control of spread of infection. If infection spreads, this can lead to immense losses over large areas of cultivated land. (ii) Screening seeds for inputs into hybridization programs, (iii) Quality control of seeds.

In another exemplary embodiment, the system 100 is used to find which pixel cluster is responsible for a classification by a neural network (e.g., a cat is present in an image), the probabilistic graphical model with a sparsity assumption on the pixels may be applied. The system 100 may pick out those pixels that most strongly drive the neural network’s decision that there is a cat in the image. Similarly, when the neural network says that a cat is absent from the image, the system 100 make sure that there is good coverage of the neural network on all parts of the image. If the system 100 finds this not to be the case, this gives an opportunity to create adversarial examples by including cat images in parts of the image that the neural network is attending to more poorly.

In another exemplary embodiment, the system 100 is used in outlier and heavy-hitter detection. The heavy-hitter detection is a group testing problem where there are n objects (milk samples, for example), and each object has a numerical value associated with it (e.g., antibiotic levels). A very small number of these objects are heavy hitters in the sense that their numerical values are outliers. For example, some of the milk samples are very high in antibiotic levels. In such example scenario, the system 100 determines heavy hitters such as antibiotic levels in the milk samples by solving the nonlinear inverse problems using the probabilistic graphical model This assumption for regularity condition is different from sparsity because each component of the vector is nonzero. So, traditional approaches that seek to exploit sparsity may not work. Such that, the nonlinear inverse problem may be formulated using the (log, exp) transformation, and with the prior representing bimodal assumption about the numerical values.

The present disclosure enables making public health screenings affordable, allowing for their comprehensive deployment. The system 100 may be implemented as a software web application which is available to guide labs in a pooling step, and to recover individual sample results using the system 100.

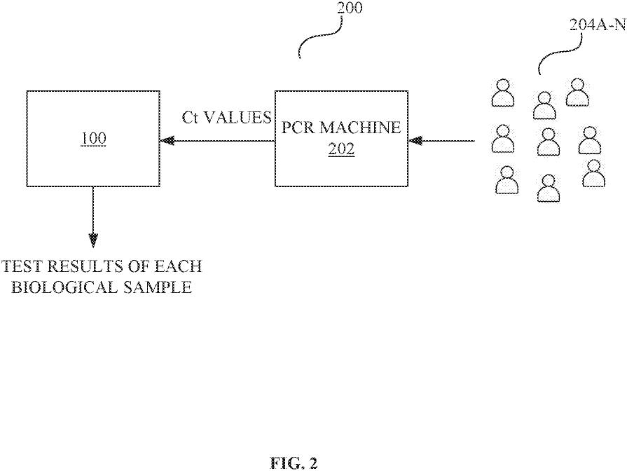

FIG. 2 is an exemplary block diagram that illustrates a use of the system 100 of FIG. 1 for detecting or retrieving test results of one or more biological samples from a polymerase chain reaction (PCR) according to some embodiments herein. The block diagram 200 includes the system 100 and PCR machine 202 that is communicatively connected with the system 100. The PCR machine 202 may be a quantitative reverse transcription polymerase chain reaction (RT-qPCR) machine. A user may select a PCR reaction plate according to a number of pools or tests to be created as per a sensing matrix or a pooling matrix. The sensing matrix or a pooling matrix may be created by the system 100 using any known pooling method or pooling scheme. The system 100 may receive a request from the user for testing of one or more biological samples 204A-N and create the pooling matrix based on a size of the one or more biological samples 204A-N. For example, the system 100 receive a request from the user for testing of 40 biological samples. The user may provide the request through a user device. The user device may be, but is not limited to, a personal computer, a notebook, a tablet, desktop computer, a laptop, a handheld device, or a mobile device. The pooling matrix includes a plurality of rows and columns. The plurality of columns indicates the number of biological samples to be tested and the plurality of rows indicates the number of tests or pools to be created for testing of the biological samples. The pooling matrix is created using the single-round combinatorial pooling method.

The user performs numbering of the biological samples 204A-N and wells of the PCR reaction plate in a matrix format, according to the pooling matrix created. Then, the user performs the pooling of the biological samples 204A-N by pipetting or transferring each of the biological sample into the different numbered wells or pools of the PCR reaction plate, according to the pooling matrix. In an embodiment, pooling of the biological samples 204A-N may involve extracting or isolating (using suitable RNA extraction kits) RNA fragments from each of the biological sample and then subsequently pipetting the extracted RNA fragment into the two or more wells or pools of the PCR reaction plate, according to the to the pooling matrix sample decoding device. RT-qPCR test may be intuitively inferred by one of ordinary skill in the art based on its name, and thus, its detailed description is omitted herein.

On performing the RT-qPCR test on each pool, the PCR machine 202 provides amplification curves corresponding to each pool. The amplification curves represent fluorescence intensity (report on a total amount of amplified DNA of the appropriate sequence) against qPCR cycles. The PCR machine 202 may derive the Ct values from the amplification curves for each pool. A smaller Ct value may indicate a greater number of copies of the viruses or microbes in the pool. Deriving of the Ct values from the amplification curves obtained by the RT-qPCR test may be intuitively inferred by one of ordinary skill in the art based on its name, and thus, its detailed description is omitted herein. The testing machine 202 may derive zero Ct values for the pool, if the pool is negative (i.e., one or more biological samples 204A-N included the corresponding pool do not include the viruses or microbes). The testing machine 202 may derive the Ct values for the pool, only if the pool is positive (i.e., one or more biological samples 204A-N in the corresponding pool include the viruses or microbes). The testing machine 202 provides the Ct values of each pool to the system 100 for retrieving or determining the test results of each biological sample.

The system 100 uses the pools that are identified as positives to retrieve the test results of each biological sample. The system 100 constructs the nonlinear equation v = f(A g(u)) based on the pooling matrix (let the pooling matrix be A) created for testing of the one or more biological samples 204A-N, the quantitative measure of viral load (let the quantitative measure of viral load be v′ of v) associated with each pool, and the quantitative measure of viral load of each biological sample. The nonlinear functions (f, g) may be at least one of, but not limited to, (log, exp), (softmax, identity), (RELU, identity), or (tanh, identity), which is applied to applied to each component of an argument vector. The system 100 solves the nonlinear equation, using the probabilistic graphical model, for each of the one or more biological samples 204A-N to retrieve the test results of each biological sample.

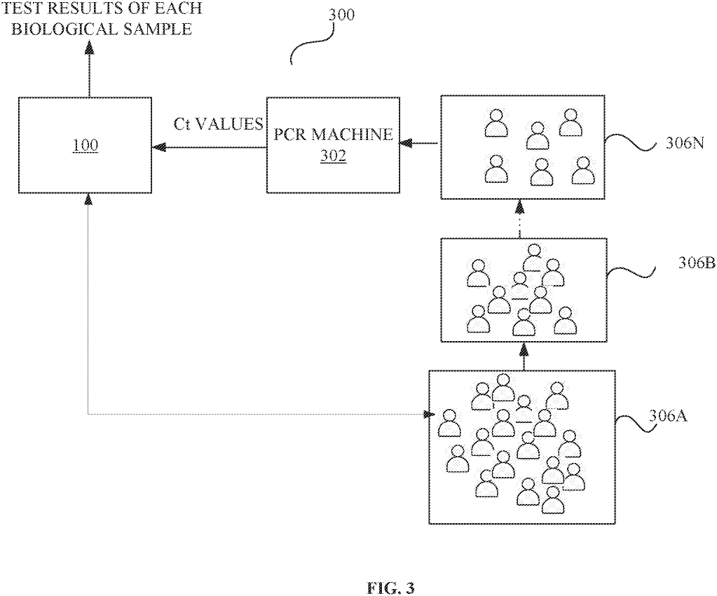

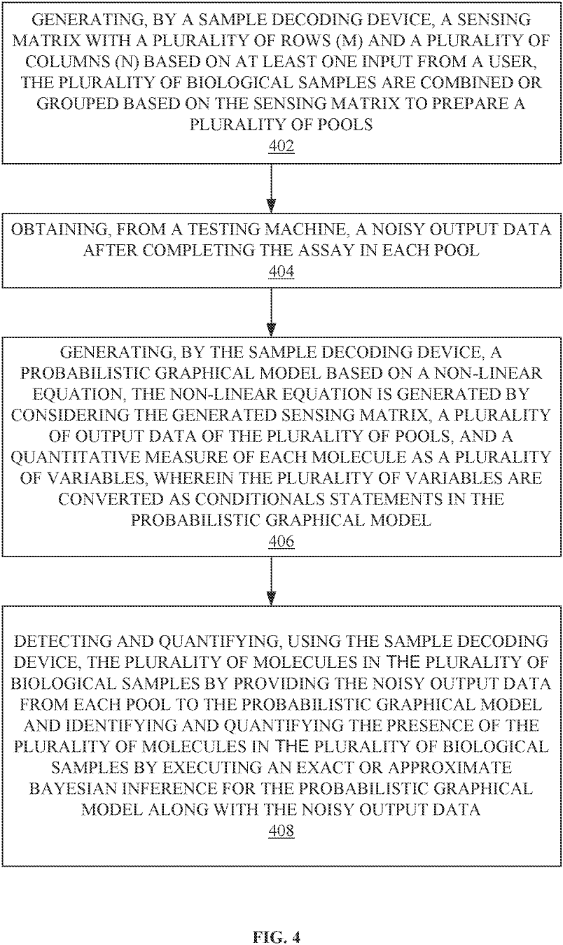

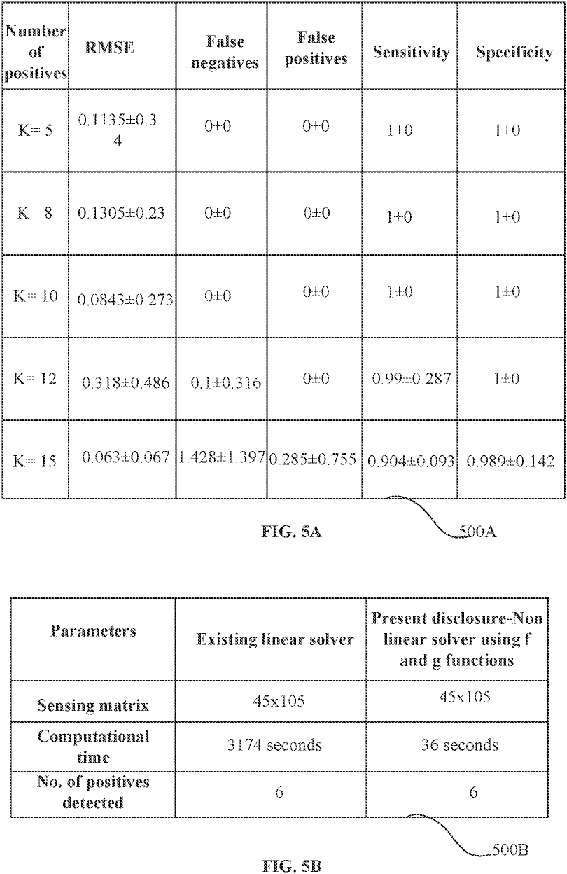

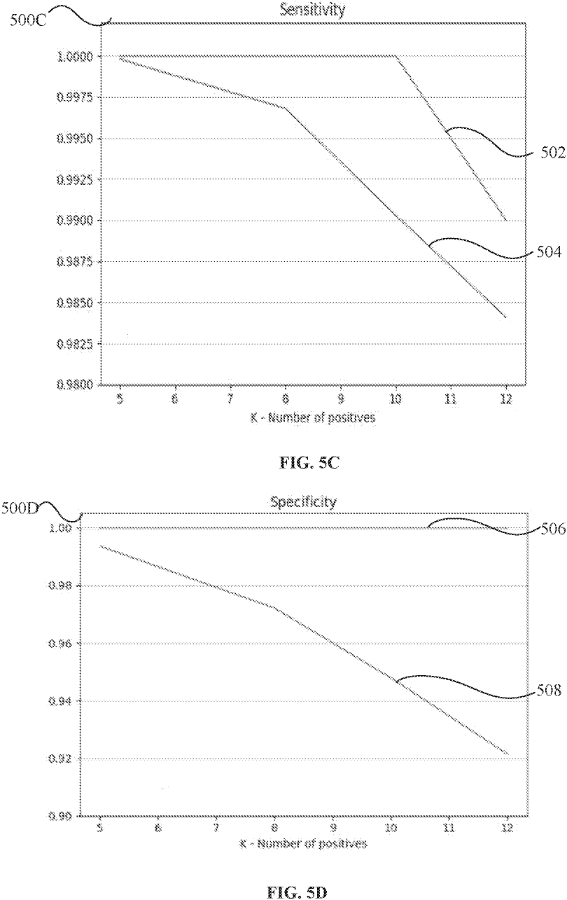

FIG. 3 is an exemplary block diagram that illustrates a use of the system 100 of FIG. 1 for detecting or retrieving test results of one or more biological samples, where a pooling matrix created from one or more iterations of pooling according to some embodiments herein. The block diagram 300 includes the system 100 and a PCR machine 302 that is communicatively connected with the system 100. The PCR machine 302 may be a quantitative reverse transcription polymerase chain reaction (RT-qPCR) machine. The system 100 may receive a request that includes a size of biological samples to be tested, from the user. The size of biological samples is a number of biological samples. Further, the system 100 may perform n (n=1,2,3,....) iterations of pooling to create a pooling matrix. In one embodiment, the system 100 (i) creates a first pooling matrix 306A based on a size of the biological samples using known pooling method, (ii) subsequently creates a second pooling matrix 306B based on the first pooling matrix 306A and (iii) thereafter creates a n^{th} pooling matrix 306N based on the second pooling matrix 306B or a previous pooling matrix. A size of the second pooling matrix 306B or number of pools of the second pooling matrix 306B is smaller than number of pools of the first pooling matrix 306A. Accordingly, a size of the n^{th} pooling matrix 306N or number of pools of the n^{th} pooling matrix 306N is smaller than the number of pools of the second pooling matrix 306B or the previous pooling matrix. A number of iterations for creating the pooling matrix may depend on the size of the biological samples to be tested. Each level of pooling obtains a compression. Repeating this multiple times obtains a multiplicative compression.

On performing the RT-qPCR test on each pool of the n^{th} pooling matrix 306N, the testing machine 302 provides amplification curves corresponding to each pool. The amplification curves represent fluorescence intensity (report on a total amount of amplified DNA of the appropriate sequence) against qPCR cycles. The testing machine 202 may derive the Ct values from the amplification curves for each pool. In some embodiments, the testing machine is used to perform the RT-qPCR test on each pool of the first pooling matrix 306A or the second pooling matrix 306B.The system 100 uses the pools that are identified as positives to retrieve the test results of each biological sample. The system 100 constructs the nonlinear equation v = f(A g(u)) based on the n^{th} pooling matrix (let the pooling matrix be A) created for testing of the one or more biological samples, the quantitative measure of viral load (let the quantitative measure of viral load be v′ of v) associated with each pool, and quantitative measure of viral load of each sample. The nonlinear functions (f, g) may be at least one of, but not limited to, (log, exp), (softmax, identity), (RELU, identity), or (tanh, identity), which is applied to applied to each component of an argument vector. The system 100 solves the nonlinear equation, using the probabilistic graphical model, for each of the one or more biological samples to retrieve the test results of each biological sample.

FIG. 4 illustrates a method for detecting and quantifying a plurality of molecules in a plurality of biological samples based on a noisy output data from an assay on each pool according to some embodiments herein. At a step 402, a sensing matrix with a plurality of rows (m) and a plurality of columns (n) is generated, using a sample decoding device, based on at least one input from a user. The plurality of biological samples are combined or grouped based on the sensing matrix to prepare a plurality of pools. The plurality of rows (m) indicate the plurality of pools to be created for testing of the plurality of biological samples. The plurality of columns (n) indicate the plurality of biological samples to be tested. The at least input includes at least one of a name of the assay, and a size of the assay, wherein the size of the assay indicates a total number of biological samples to be tested and a number of biological samples estimated as positive out of the total number of biological samples. In some embodiments, the sensing matrix is created using a Steiner triples system.

At a step 404, a noisy output data after completing the assay in each pool, is obtained from a testing machine. The testing machine may be selected from a group including of a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy. The noisy output data is an output data with noise from each pool.

At step 406, a probabilistic graphical model is generated, using the sample decoding device, based on a non-linear equation for detecting and quantifying the plurality of molecules in the plurality of biological samples. The non-linear equation is generated by based on a plurality of variables that include the generated sensing matrix, a plurality of output data of the plurality of pools, and a quantitative measure of each molecule. The plurality of variables are converted as conditionals statements in the probabilistic graphical model. The nonlinear equation includes v = f(A g(u)), wherein (a) A is the sensing matrix with the plurality of rows (m) and the plurality of columns (n); (b) u is a column vector of dimension n, wherein the n indicates a number of the plurality of biological samples to be tested, wherein detection of the column vector (u) enables to detect the presence or absence of the molecules in the plurality of biological samples and quantify the molecules if the molecules are present in the plurality of biological samples; (c) v is a vector of dimension m, wherein v is considered as the output data from each pool and v′ is considered as the noisy output data from each pool; (d) g is a nonlinear vector-valued function of n variables; and (e) f is a nonlinear vector-valued function of m variables. In some embodiment, the probabilistic graphical model is generated by (i) writing, using a probabilistic programming language, the nonlinear equation, (ii) converting observed variables into conditioning statements, (iii) generating the probabilistic graphical model based on the nonlinear equation (which in probabilistic programming language) and the conditioning statements. The observed variables include the generated sensing matrix, the plurality of output data of the plurality of pools, and the quantitative measure of each molecule. The observed values for the conditioned variables are fed at a time of Markov chain Monte Carlo (MCMC) inference.

At step 408, the plurality of molecules in the plurality of biological samples are detected and quantified, using the sample decoding device by providing the noisy output data from each pool to the probabilistic graphical model and identifying and quantifying the presence of the plurality of molecules in the plurality of biological samples by executing exact or approximate Bayesian inference for the probabilistic graphical model along with the noisy output data.

The method further includes detecting a condition of interest based on the detected and quantified molecules in the plurality of biological samples. The condition of interest may be an infectious disease, cancer, a genetic disease, an inflammation condition, a metabolic syndrome, cardiac disease, or diabetes.

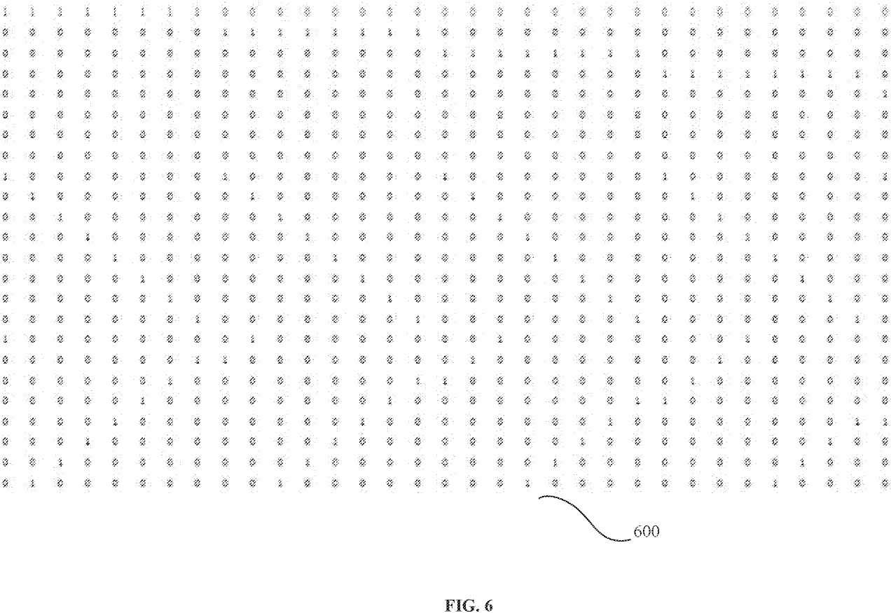

FIG. 5A is a table 500A of experimental results that illustrates an accuracy of the system 100 of FIG. 1 in detecting and quantifying the plurality of molecules in the plurality of biological samples according to some embodiments herein. In the table 500A, k indicates a number of positives that are identified from a given biological samples. Accuracy metrics of the sample decoding device 106 is identified by running the sample decoding device 106 on 45×105 sensing matrix using synthetic data and averaged over 10 runs. With reference to the table 500A, the system 100 of FIG. 1 has a sensitivity of 0.904 to 1 and specificity of 0.989 to 1. Sensitivity is an ability of a test to correctly identify patients with a disease. Specificity: the ability of a test to correctly identify people without the disease.

With reference to FIG. 5A, FIG. 5B is a table 500B of experimental results that illustrates a computational efficiency of the system 100 of FIG. 1 in detecting and quantifying the plurality of molecules in the plurality of biological samples according to some embodiments herein. With reference to table 500B, the system 100 of FIG. 1 detects 6 samples as positive out of 105 samples in 36 seconds. Positive indicates that the sample includes a molecule of interest (e.g., virus). The system 100 detects the molecules of interest in the given samples by executing the exact or approximate Bayesian inference for the probabilistic graphical model along with the generated synthetic data for 45*105 sensing matrix. The probabilistic graphical model specifies the nonlinear functions f and g as log and exp respectively during executing the exact or approximate Bayesian inference. Whereas, an existing linear solver or compressed sensing solver detects 6 samples as positive out of 105 samples in 3174 seconds. It is observed that the system 100 of FIG. 1 is 88.16 times faster than the existing linear solver while running on a same data such as a 45*105 sensing matrix.

With reference to FIGS. 5A and 5B, FIG. 5C is an exemplary graphical representation 500C that illustrates sensitivity of the system 100 of FIG. 1 in detecting and quantifying the plurality of molecules in the plurality of biological samples in comparison with existing linear solver or compressed sensing solver according to some embodiments herein. In the exemplary graphical representation 500C, a number of positives are plotted in an X-axis and a sensitivity score is plotted in an Y-axis. In the exemplary graphical representation 500C, a solid line 502 illustrates the sensitivity of the system 100 in detecting the number of positives in the given samples. In the exemplary graphical representation 500C, a solid line 504 illustrates the sensitivity of the existing linear solver or compressed sensing solver in detecting the number of positives in the given samples. It is observed that the sensitivity of the existing linear solver declines when compared to the system 100.

With reference to FIGS. 5A-5C, FIG. 5D is an exemplary graphical representation that illustrates specificity of the system of FIG. 1 in detecting and quantifying the plurality of molecules in the plurality of biological samples in comparison with existing linear solver or compressed sensing solver according to some embodiments herein. In the exemplary graphical representation 500D, a number of positives are plotted in an X-axis and a specificity score is plotted in an Y-axis. In the exemplary graphical representation 500D, a solid line 506 illustrates the specificity of the system 100 in detecting the number of positives in the given samples. In the exemplary graphical representation 500D, a solid line 508 illustrates the specificity of the existing linear solver or compressed sensing solver in detecting the number of positives in the given samples. It is observed that the specificity of the existing linear solver declines when compared to the system 100. The system 100 has the specificity score of 1.

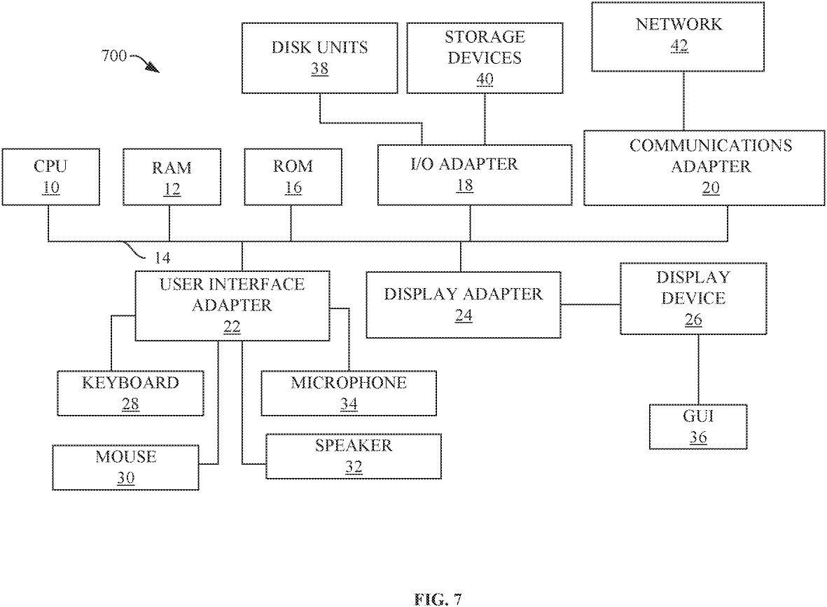

FIG. 6 is an exemplary 24*64 sensing matrix 600 that is generated using the system of FIG. 1 according to some embodiments herein. The exemplary 24*64 sensing matrix is generated by the sample decoding device 106, using a pooling technique, based on the at least one input that includes a name of the assay (e.g., PCR), and a size of the assay (e.g., 64), and a number of biological samples estimated as positive out of the total number of biological samples. The exemplary 24*64 sensing matrix includes 24 rows and 64 columns. The exemplary 24*64 sensing matrix includes a plurality of zero (0) entries and a plurality of non-zero (1) entries. The values 1 with respect to each column indicates the pools for including the biological sample corresponding to each column. The number of rows of the exemplary sensing matrix indicate 24 pools to be created for testing the plurality of biological samples. The number of columns of the sensing matrix indicate 64 biological samples to be tested.

FIG. 7 is a schematic diagram of computer architecture of a computing device or a molecular computer 700, in accordance with the embodiments herein. A representative hardware environment for practicing the embodiments herein is depicted in FIG. 7, with reference to FIGS. 1 through 6. This schematic drawing illustrates a hardware configuration of a server/computer system/computing device/molecular computer in accordance with the embodiments herein. The system 100 of FIG. 1 may use the computing device or the molecular computer 700 for detecting and quantifying a plurality of molecules in a plurality of biological samples according to the embodiments herein. The computing device or the molecular computer 700 includes at least one processing device CPU 10 that may be interconnected via system bus 14 to various devices such as a random-access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents employed herein is for the purpose of description and not of limitation. Therefore, while the embodiments herein have been described in terms of preferred embodiments, those skilled in the art will recognize that the embodiments herein can be practiced with modification within the scope.

What is claimed is:

1. A system for detecting and quantifying a plurality of molecules in a plurality of biological samples based on a noisy output data from an assay on each pool, wherein the system comprising:
a memory that stores a set of instructions;
a processor that is configured to execute the set of instructions for performing one or more operations, the processor is configured to
generate, using a sample decoding device, a sensing matrix with a plurality of rows (m) and a plurality of columns (n) based on at least one input from a user, wherein the plurality of biological samples are combined or grouped based on the sensing matrix to generate a plurality of pools;
obtain, from a testing machine, a noisy output data after completing the assay in each pool, wherein the noisy output data is an output data with noise from each pool;
generate, using the sample decoding device, a probabilistic graphical model based on a non-linear equation for detecting and quantifying the plurality of molecules in the plurality of biological samples, wherein the non-linear equation is generated based on a plurality of variables that comprise the generated sensing matrix, a plurality of output data of the plurality of pools, and a quantitative measure of each molecule, wherein the plurality of variables are converted as conditionals statements in the probabilistic graphical model; and
detect and quantify, using the sample decoding device, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data.

2. The system of claim 1, wherein the processor is configured to detect a condition of interest based on the detected and quantified molecules in the plurality of biological samples, wherein the condition of interest comprises at least one of an infectious disease, cancer, a genetic disease, an inflammation condition, a metabolic syndrome, cardiac disease, or diabetes.

3. The system of claim 1, wherein the testing machine is a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy.

4. The system of claim 1, wherein the nonlinear equation comprises v = f(A g(u)), wherein the,
(a) A is the sensing matrix with the plurality of rows (m) and the plurality of columns (n);
(b) u is a column vector of dimension n, wherein the n indicates a number of the plurality of biological samples to be tested, wherein detection of the column vector (u) enables to detect the presence or absence of the plurality of molecules in the plurality of biological samples and quantify the plurality of molecules if the molecules are present in the plurality of biological samples;
(c) v is a vector of dimension m, wherein v is considered as the output data from each pool and v′ is considered as the noisy output data of the output data from each pool;
(d) g is a nonlinear vector-valued function of n variables; and
(e) f is a nonlinear vector-valued function of m variables.

5. The system of claim 1, wherein executing the exact or approximate Bayesian inference comprises systemically specifying prior and regulatory conditions for the probabilistic graphical model.

6. The system of claim 4, wherein the processor is configured to
convert a noisy linear inverse problem into a noisy nonlinear inverse problem when there are multiples orders of nonzero entries in the sensing matrix; and
construct the nonlinear equation v = log(A e^{u}) by considering f and g as log and exp functions instead of considering f and g as identity functions, wherein the nonzero entries indicate that each sample in the plurality of columns (n) of the sensing matrix (A) represents at least one signal.

7. The system of claim 1, wherein the processor is configured to perform n (n=1,2,3,....) iterations to create the sensing matrix for obtaining compression in pooling by (i) creating a first sensing matrix based on a size of the assay, (ii) subsequently creating a second sensing matrix based on the first sensing matrix, and (iii) thereafter creating a n^{th} sensing matrix based on the second sensing matrix or a previous sensing matrix, wherein a size of the second sensing matrix or a number of pools of the second sensing matrix is smaller than a number of pools of the first sensing matrix.

8. The system of claim 1, wherein (i) the plurality of rows (m) indicate the plurality of pools to be created for testing of the plurality of biological samples; and (ii) the plurality of columns (n) indicate the plurality of biological samples to be tested.

9. The system of claim 1, wherein the at least input comprises at least one of a name of the assay, and a size of the assay, wherein the size of the assay indicates a total number of biological samples to be tested and a number of biological samples estimated as positive out of the total number of biological samples.

10. A processor implemented method for detecting and quantifying a plurality of molecules in a plurality of biological samples based on a noisy output data from an assay on each pool, wherein the method comprising:
generating, using a sample decoding device, a sensing matrix with a plurality of rows (m) and a plurality of columns (n) based on at least one input from a user, wherein the plurality of biological samples are combined or grouped based on the sensing matrix to
obtaining, from a testing machine, a noisy output data after completing the assay in each pool, wherein the noisy output data is an output data with noise from each pool;
generating, using the sample decoding device, a probabilistic graphical model based on a non-linear equation for detecting and quantifying the plurality of molecules in the plurality of biological samples, wherein the non-linear equation is generated based on a plurality of variables that comprise the generated sensing matrix, a plurality of output data of the plurality of pools, and a quantitative measure of each molecule, wherein the plurality of variables are converted as conditionals statements in the probabilistic graphical model; and
detecting and quantifying, using the sample decoding device, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data.

11. The processor implemented method of claim 10, wherein the method further comprises detecting a condition of interest based on the detected and quantified molecules in the plurality of biological samples, wherein the condition of interest comprises at least one of an infectious disease, cancer, a genetic disease, an inflammation condition, a metabolic syndrome, cardiac disease, or diabetes.

12. The processor implemented method of claim 10, wherein the testing machine is a polymerase chain reaction (PCR) machine, a high-performance liquid chromatography (HPLC), microarray screens, a next generation sequencing (NGS) device, a mass spectrometry, a nuclear magnetic resonance (NMR) spectroscopy, or a Raman spectroscopy.

13. The processor implemented method of claim 10, wherein the nonlinear equation comprises v = f(A g(u)), wherein the,
(a) A is the sensing matrix with the plurality of rows (m) and the plurality of columns (n);
(b) u is a column vector of dimension n, wherein the n indicates a number of the plurality of biological samples to be tested, wherein detection of the column vector (u) enables to detect the presence or absence of the plurality of molecules in the plurality of biological samples and quantify the plurality of molecules if the molecules are present in the plurality of biological samples;
(c) v is a vector of dimension m, wherein v is considered as the output data from each pool and v′ is considered as the noisy output data of the output data from each pool;
(d) g is a nonlinear vector-valued function of n variables; and
(e) f is a nonlinear vector-valued function of m variables.

14. The processor implemented method of claim 10, wherein executing the exact or approximate Bayesian inference comprises systemically specifying prior and regulatory conditions for the probabilistic graphical model.

15. The processor implemented method of claim 13, wherein the method further comprises
convert a noisy linear inverse problem into a noisy nonlinear inverse problem when there are multiples orders of nonzero entries in the sensing matrix; and
construct the nonlinear equation v = log(A e^{u}) by considering f and g as log and exp functions instead of considering f and g as identity functions, wherein the nonzero entries indicate that each sample in the plurality of columns (n) of the sensing matrix (A) represents at least one signal.

16. The processor implemented method of claim 10, wherein the method performs n (n=1,2,3,....) iterations to create the sensing matrix for obtaining compression in pooling by (i) creating a first sensing matrix based on a size of the assay, (ii) subsequently creating a second sensing matrix based on the first sensing matrix, and (iii) thereafter creating a n^{th} sensing matrix based on the second sensing matrix or a previous sensing matrix, wherein a size of the second sensing matrix or a number of pools of the second sensing matrix is smaller than a number of pools of the first sensing matrix.

17. The processor implemented method of claim 10, wherein (i) the plurality of rows (m) indicate the plurality of pools to be created for testing of the plurality of biological samples; and (ii) the plurality of columns (n) indicate the plurality of biological samples to be tested.

18. The processor implemented method of claim 10, wherein the at least input comprises at least one of a name of the assay, and a size of the assay, wherein the size of the assay indicates a total number of biological samples to be tested and a number of biological samples estimated as positive out of the total number of biological samples.

19. A one or more non-transitory computer-readable storage mediums storing the one or more sequences of instructions, which when executed by the one or more processors, causes to perform a method of detecting and quantifying a plurality of molecules in a plurality of biological samples based on a noisy output data from an assay on each pool, wherein the method comprises:
generating, using a sample decoding device, a sensing matrix with a plurality of rows (m) and a plurality of columns (n) based on at least one input from a user, wherein the plurality of biological samples are combined or grouped based on the sensing matrix to generate a plurality of pools;
obtaining, from a testing machine, a noisy output data after completing the assay in each pool, wherein the noisy output data is an output data with noise from each pool;
generating, using the sample decoding device, a probabilistic graphical model based on a non-linear equation for detecting and quantifying the plurality of molecules in the plurality of biological samples, wherein the non-linear equation is generated based on a plurality of variables that comprise the generated sensing matrix, a plurality of output data of the plurality of pools, and a quantitative measure of each molecule, wherein the plurality of variables are converted as conditionals statements in the probabilistic graphical model; and
detecting and quantifying, using the sample decoding device, the plurality of molecules in the plurality of biological samples by providing the noisy output data from each pool to the probabilistic graphical model and identifying and quantifying the presence of the plurality of molecules in the plurality of biological samples by executing an exact or approximate Bayesian inference for the probabilistic graphical model along with the noisy output data.
